(19) United States
(12) Patent Application Publication
(10) Pub. No.: US 2026/0347788 A1
(43) Pub. Date: Nov. 5, 2026

(54) HUMAN GASTROINTESTINAL ORGANOIDS, METHODS OF PRODUCING SAME, AND USES THEREOF

(71) Applicant: VALORISATION HSJ, LIMITED PARTNERSHIP, Québec (CA)

(72) Inventors: Taehun SONG, Montréal (CA); Gregor ANDELFINGER, Montréal (CA)

(21) Appl. No.: 19/488,622

(22) PCT Filed: May 28, 2024
(86) PCT No.: PCT/CA2024/050706

§ 371 Date: Nov. 27, 2025

Related U.S. Application Data
(60) Provisional application No. 63/504,764, filed on May 29, 2023.

(51) Int. Cl. C12N 5/071

(52) U.S. Cl. CPC C12N 5/0679; C12N 2500/02; C12N 2500/30; C12N 2500/32; C12N 2500/90; C12N 2501/11; C12N 2501/119; C12N 2501/16; C12N 2501/415; C12N 2502/081; C12N 2502/1347; C12N 2513/00

(57) ABSTRACT

One of the primary challenges of prolonged in vitro culture of pluripotent stem cell-derived tissue or organoid is to mature it further down the developmental timeline to have the functional features of the organ while limiting the increase in size. Nutrient/gas exchange diminishes towards the center of the organoid as it grows, and vascularization would normally be required to overcome this limitation. The present application relates to an in vitro method, which does not require vascularization, for generating human gastrointestinal organoids (HGIOs) such as human intestinal organoids (HIOs) with contractile activity. This method involves culturing immature HGIOs in a non-adherent environment in a culture medium free or substantially free of epidermal growth factor (EGF) for at least one week. HGIOs generated by this method mimic intestinal structures and exhibit peristaltic movement and contractile activity. They may be used for assessing the effects of new therapeutic agents on intestinal function.

### CROSS REFERENCE TO RELATED APPLICATIONS

The present application claims the benefit of U.S. provisional patent application Ser. No. 63/504,764 filed on May 29, 2023, which is incorporated herein by reference in its entirety.

### TECHNICAL FIELD

The present disclosure generally relates to the field of organoids, and more particularly to the generation of human gastrointestinal organoids in vitro.

### BACKGROUND ART

The study of human gastrointestinal biology in healthy and diseased conditions has always been challenging. Primary obstacles have included limited tissue accessibility, inadequate in vitro maintenance and ethical constraints. The development of three-dimensional organoid cultures has been a significant breakthrough in the field. Intestinal organoids are self-organized three-dimensional structures that partially mimic the structure, cell heterogeneity and cell behavior of the original tissue in vitro. This includes studying the capacity of intestinal stem cells to self-renew and differentiation towards various epithelial lineages. Therefore, over the past decade, the use of human organoid cultures has been instrumental to model human intestinal development, homeostasis, disease, and regeneration. Different types of intestinal organoids can be derived from pluripotent stem cells (PSC) or from adult somatic intestinal stem cells (ISC) each with different features.

One of the primary challenges of prolonged in vitro culture of pluripotent stem cell derived tissue, or organoid, is to mature it further down the developmental timeline to have the functional features of the organ while limiting the increase in size. Nutrient/gas exchange diminishes towards the center of the organoid as it grows, and vascularization would normally be required to overcome this limitation. Many efforts are being made to engineer effective means of in vitro vascularization¹⁻³.

Watson et al.¹¹ and Workman et al.⁸ disclose a method for generating human pluripotent stem cell (hPSC)-derived intestinal tissue with a functional enteric nervous system (ENS) that comprises engrafting hPSC-derived Human Intestinal Organoids (HIO) into mice and allowed them to grow for 6-10 weeks in vivo with the help of vasculature of mice. Because it requires engraftment into mice, this method is rather complex and expensive.

There is thus a need for novel approaches for the generation of functional human intestinal organoids entirely in vitro.

The present description refers to a number of documents, the content of which is herein incorporated by reference in their entirety.

### SUMMARY

In various aspects and embodiments, the present disclosure provides the following items 1 to 42:
1. An in vitro method for generating human gastrointestinal organoid (HGIO) having contractile
   activity comprising:
   providing an immature HGIO;
   culturing the immature HGIO in a non-adherent environment in a culture medium that is free or substantially free of epidermal growth factor (EGF) under conditions suitable for HGIO growth for a period of at least 1 week, thereby obtaining (HGIO) having contractile activity;
   collecting the HGIO.
2. The method of item 1, wherein the culture medium that is free or substantially free of any growth factor of the EGF family.
3. The method of item 1 or 2, wherein the culturing is for a period of at least two weeks.
4. The method of item 1 or 2, wherein the culturing is for a period of four to five weeks.
5. The method of any one of items 1 to 4, wherein the culture medium comprises Dulbecco's Modified Eagle Medium/Nutrient Mixture F-12.
6. The method of item 5, wherein the culture medium further comprises a B-27 supplement.
7. The method of item 5 or 6, wherein the culture medium further comprises a source of L-glutamine.
8. The method of any one of 5 to 7, wherein the culture medium further comprises 4-(2-hydroxyethyl)-1-piperazineethanesulfonic acid (HEPES).
9. The method of any one of 5 to 8, wherein the culture medium further comprises one or more antibiotics.

10. The method of item 9, wherein the one or more antibiotics comprise penicillin and streptomycin.
11. The method of any one of items 1 to 10, wherein the conditions suitable for HIO growth comprise incubation at a temperature of about 37 °C.
12. The method of any one of items 1 to 11, wherein the culture medium is replaced with fresh medium at least every 48 to 96 hours.
13. The method of item 12, wherein the culture medium is replaced with fresh medium at least every 48 hours.
14. The method of any one of items 1 to 13, wherein the culturing is performed in an ultra low attachment (ULA) culture flask.
15. The method of any one of items 1 to 14, wherein the immature HGIO has a diameter that is 1 mm or less.
16. The method of item 15, wherein the immature HGIO has a diameter that is 0.7 mm or less.
17. The method of any one of items 1 to 16, wherein the immature HIO are derived from embryonic or pluripotent stem cells.
18. The method of item 17, wherein the pluripotent stem cells are human induced pluripotent stem cells (hiPSC).
19. The method of any one of items 1 to 18, further comprising generating the immature HGIO.
20. The method of item 19, wherein generating the immature HGIO comprises:
   (i) culturing pluripotent stem cells in an endoderm differentiation medium supplemented with Activin A until obtention of confluent monolayer of cells;
   (ii) culturing the confluent monolayer of cells in a mid-hindgut differentiation medium supplemented with a Fibroblast growth factor (FGF) and a Wnt pathway activator until obtention of free-floating spheroids;
   (iii) mixing the spheroids with a first biocompatible gel supplemented with epidermal growth factor (EGF)
   (iv) culturing the gel-spheroid mixture in an intestinal basal medium supplemented with EGF until obtention of primary gastrointestinal organoids;
   (v) mixing the primary gastrointestinal organoids and enteric neural cell precursors with a second biocompatible gel; and
   (vi) culturing the mixture of (v) in an intestinal basal medium supplemented with EGF until obtention of the immature HGIO.
21. The method of item 20, wherein the FGF is FGF4.
22. The method of item 20 or 21, wherein the Wnt pathway activator is CHIR99021.
23. The method of any one of items 20 to 22, wherein the first and/or second biocompatible gel comprises extracellular matrix. 24. The method of any one of items 20 to 23, wherein the first and/or second biocompatible gel is a solubilized basement membrane matrix secreted by Engelbreth-Holm-Swarm (EHS) mouse sarcoma cells (Matrigel™).
25. The method of any one of items 20 to 24, wherein the enteric neural cell precursors comprise vagal neural crest cells.
26. The method of any one of items 1 to 25, wherein the human gastrointestinal organoid is a human intestinal organoid (HIO).
27. An in vitro human gastrointestinal organoid (HGIO) having contractile peristaltic-like activity and comprising:
   smooth muscle cells,
   interstitial cells of cajal,
   epithelial cells,
   crypt-villi structures, and optionally
   enteric neuron cells.
28. The HGIO of item 27, wherein the HGIO comprises:
   an inner layer comprising the crypt-villi structures comprising epithelial cells and intestinal stem cells;
   a mid-layer comprising connective tissue cells, and, optionally, enteric neuron cells;
   an outer layer comprising (i) a first (e.g., inner) comprising smooth muscle cells and (ii) a second (e.g., outer) sublayer comprising interstitial cells of cajal and, optionally (iii) enteric neuron cells.
29. The HGIO of item 27 or 28, wherein the smooth muscle cells express TAGLN and/or SMTN.
30. The HGIO of any one of items 27 to 29, wherein the interstitial cells of cajal express KIT.
31. The HGIO of any one of items 27 to 30, wherein the epithelial cells express CDX2.
32. The HGIO of any one of items 27 to 31, wherein the crypt structures express MKI67.
33. The HGIO of any one of items 27 to 32 wherein the enteric neuron cells express ELAVL4 and/or TUBB3.
34. The HGIO of any one of items 27 to 33, wherein the epithelial cells face the inside of the HGIO.
35. The HGIO of any one of items 27 to 34, wherein the enteric neuron cells form ganglia.
36. The HGIO of item 35, wherein the ganglia have a size of at least 10 μm.
37. The HGIO of item 36, wherein the ganglia have a size of 15 μm to 50 μm.
38. The HGIO of any one of items 27 to 37, wherein the smooth muscle cells form one or more layers.
39. The HGIO of item 38, wherein the one or more smooth muscle layer(s) have a thickness of at least 30 μm.
40. The HGIO of item 39, wherein the one or more smooth muscle layer(s) have a thickness of 40 μm to 200 μm.
41. The HGIO of any one of items 27 to 40, wherein the HGIO is a human intestinal organoid (HIO).
42. The HGIO of any one of items 27 to 41, wherein the HGIO are obtained according to the method of any one of items 1 to 26.

Other objects, advantages and features of the present disclosure will become more apparent upon reading of the following non-restrictive description of specific embodiments thereof, given by way of example only with reference to the accompanying drawings.

### BRIEF DESCRIPTION OF DRAWINGS

In the appended drawings:

FIG. 1 depicts a schematic representation of the time-line of human intestinal organoid derivation and the experimental conditions.

FIG. 2 shows images of the maturation of HIOs in different biochemical and biophysical conditions. Top row: bright field images of HIOs in culture. Middle and Bottom row: immunofluorescence staining of cryosectioned HIOs. White arrowheads indicate the location of organoids in the middle panel. 100 ng/ml human EGF in ULA, n=12.0 ng/ml human EGF in Matrigel™, n=12.0 ng/ml human EGF in ULA, n=50.

FIGS. 3A-D show the contractile activity of HIOs and the conducive conditions of smooth muscle differentiation. FIG. 3A: Contractile activity of HIOs derived using different experimental conditions. FIG. 3B: Immunostain of organoids maintained in different concentrations of growth factor. FIG. 3C: Immunostain of mesenchymal cells in each experimental condition. FIG. 3D: Proportion of contractile organoids at week 4 of HIO in each condition. 100 ng/ml human EGF in ULA, n=12.0 ng/ml human EGF in Matrigel™, n=12.0 ng/ml human EGF in ULA, n=50.

FIGS. 4A-D depict immunostain for markers of enteric neuron (FIG. 4A), intestinal identity (FIG. 4B), smooth muscle (FIG. 4C) and crypt-villi (FIG. 4D) in week 5 HIOs incorporated with VNCC.

FIGS. 5A-D show the response of in vitro matured contractile HIO/ENS to promotility drugs. FIG. 5A: overview of the effects of the compound tested. FIG. 5B: effect of 10 μM metoclopramide on contractile activity of in vitro matured HIOs. FIG. 5C: effect of control vehicle (left) and acetylcholine (right) on contractile activity of in vitro matured HIOs. FIG. 5D: effect of various doses of prucalopride (left graph), pyridostigmine (middle graph) and metoclopramide (right graph) on contractile activity of in vitro matured HIOs. Size reduction of organoids due to sustained muscle contraction was measured by bright field imaging. Student's t-test, p-value as shown in figures.

FIGS. 6A-C show the comparison of tissue maturity of in vitro matured contractile HIO/ENS according to the present disclosure (Disclosure) to gut organoid described Uchida et al.¹² and human fetal intestine (Intestine). Student's t-test, p-value as shown in figures.

FIG. 6A: Muscle thickness (left graph) and number of cells constituting the smooth muscle (right graph). Quantified by measuring, in cross sections of each sample, continuous layer of cells positive for smooth muscle marker and counting number of nuclei in a straight line through the smooth muscle from lumen to outside. Diagram on the right visualizes the difference between smooth muscle tissue consisted of continuous layer of cells (bottom) versus individual smooth muscle cells (top).

FIG. 6B: Size and number of cells constituting of neuronal ganglia (no clusters of multiple neurons were observable in Uchida et al.). Quantified by measuring shortest distance of a ganglion and counting the number of nuclei within a ganglion. Diagram on the right depicts the structure of an enteric neuronal ganglion.

FIG. 6C: Specificity of distribution of Interstitial Cells of Cajal (ICC). Quantified by measuring the fluorescence intensity of anti-KIT antibody (marker of ICC) in a straight line through the intestine from lumen to outside.

FIGS. 7A-D show gene expression at a single cell level for HIO made with two different experimental conditions using single-cell RNA sequencing technique. FIGS. 7A-B show intestinal cell types identified in HIO made with experimental condition 100 ng/ml EGF in Matrigel™ and the expression of genes known to identify in each cell type. MUC1 expression was 0 thus not displayed on the graph. FIGS. 7C-D show intestinal cell types identified in HIO made with experimental condition 0 ng/ml EGF in ULA and expression of genes known to identify in each cell type.

### DETAILED DISCLOSURE

The use of the terms “a” and “an” and “the” and similar referents in the context of describing the technology (especially in the context of the following claims) are to be construed to cover both the singular and the plural, unless otherwise indicated herein or clearly contradicted by context.

The terms “comprising”, “having”, “including”, and “containing” are to be construed as open-ended terms (i.e., meaning “including, but not limited to”) unless otherwise noted.

All methods described herein can be performed in any suitable order unless otherwise indicated herein or otherwise clearly contradicted by context.

The use of any and all examples, or exemplary language (“e.g.”, “such as”) provided herein, is intended merely to better illustrate embodiments of the claimed technology and does not pose a limitation on the scope unless otherwise claimed.

No language in the specification should be construed as indicating any non-claimed element as essential to the practice of embodiments of the claimed technology.

Herein, the term “about” has its ordinary meaning. The term “about” is used to indicate that a value includes an inherent variation of error for the device or the method being employed to determine the value, or encompass values close to the recited values, for example within 10% of the recited values (or range of values).

Recitation of ranges of values herein are merely intended to serve as a shorthand method of referring individually to each separate value falling within the range, unless otherwise indicated herein, and each separate value is incorporated into the specification as if it were individually recited herein. All subsets of values within the ranges are also incorporated into the specification as if they were individually recited herein.

Where features or aspects of the disclosure are described in terms of Markush groups or list of alternatives, those skilled in the art will recognize that the disclosure is also thereby described in terms of any individual member, or subgroup of members, of the Markush group or list of alternatives.

Unless specifically defined otherwise, all technical and scientific terms used herein shall be taken to have the same meaning as commonly understood by one of ordinary skill in the art (e.g., in stem cell biology, cell culture, molecular genetics, immunology, immunohistochemistry, protein chemistry, and biochemistry).

Unless otherwise indicated, the recombinant protein, cell culture, and immunological techniques utilized in the present disclosure are standard procedures, well known to those skilled in the art. Such techniques are described and explained throughout the literature in sources such as, J. Perbal, A Practical Guide to Molecular Cloning, John Wiley and Sons (1984), J. Sambrook et al., Molecular Cloning: A Laboratory Manual, Cold Spring Harbour Laboratory Press (1989), T. A. Brown (editor), Essential Molecular Biology: A Practical Approach, Volumes 1 and 2, IRL Press (1991), D. M. Glover and B. D. Hames (editors), DNA Cloning: A Practical Approach, Volumes 1-4, IRL Press (1995 and 1996), and F. M. Ausubel et al. (editors), Current Protocols in Molecular Biology, Greene Pub. Associates and Wiley-Interscience (1988, including all updates until present), Ed Harlow and David Lane (editors) Antibodies: A Laboratory Manual, Cold Spring Harbour Laboratory, (1988), and J. E. Coligan et al. (editors) Current Protocols in Immunology, John Wiley & Sons (including all updates until present).

In the studies described herein, the present inventors have developed an entirely in vitro method for generating human intestinal organoids (HIOs) presenting spontaneous peristaltic-like contractile activities and more faithful intestinal anatomical features than previously described methods. Immature HIOs were cultured in suspension and in a medium free of epidermal growth factor (EGF). Also, during the in vitro culture, the HIOs were kept intentionally small.

The present disclosure provides an in vitro method for generating human gastrointestinal organoid (HGIO), such as human intestinal organoid (HIO), having contractile activity comprising:
providing an immature HGIO (e.g., HIO);
culturing the immature HGIO (e.g., HIO) in a non-adherent environment in a culture medium that is free or substantially free of exogenous human epidermal growth factor (EGF) under conditions suitable for HGIO (e.g., HIO) growth for a period of at least 1 week, for example at least 10, 11, 12, 13 or 14 days, thereby obtaining HGIO (e.g., HIO) having contractile activity;
collecting the HGIO (e.g., HIO).

The term “gastrointestinal organoid” (or GI organoid) as used herein refers to a tissue structure that at least partially recapitulates the anatomical structures and functions (specifically, for example, a motile movement function or contractile activity) of an organ of the gastrointestinal tract (e.g., stomach, small intestine, colon) of the origin organism of cells, and in particular, an intestine of a mammal such as a human. It includes gastric organoids and intestinal organoids. In an embodiment, the GI organoid is an intestinal organoid.

The term “intestinal organoid” (or “gut organoid”) as used herein refers to a tissue structure that at least partially recapitulates the anatomical structures and functions (specifically, for example, a motile movement function or contractile activity) of an intestine of the origin organism of cells, and in particular, an intestine of a mammal such as a human.

The term “free or substantially free of exogenous human epidermal growth factor (EGF)” means that the medium does not contain any EGF (or any biologically active fragment/variant thereof) from an external source (i.e., exogenous EGF added to the medium), or contains exogenous EGF at a level that is too low to exert any biological activity on the HGIOs (e.g., to promote cell proliferation). In an embodiment, the level of exogenous human EGF is less than 20 ng/ml. In an embodiment, the level of exogenous human EGF is 10 ng/ml or less. In another embodiment, the level of exogenous human EGF is 1 ng/ml or less. In another embodiment, the level of exogenous human EGF is 0.1 ng/ml or less. In another embodiment, the immature HGIOs (e.g., HIOs) are cultured in a culture medium that is free of exogenous human EGF.

In another embodiment, the immature HGIOs (e.g., HIOs) are cultured in a culture medium that is free or substantially free of at least one other exogenous growth factor of the EGF family. In addition to EGF, members of the EGF family include Heparin-binding EGF-like growth factor (HB-EGF), transforming growth factor-α (TGF-α), Amphiregulin (AR), Epiregulin (EPR), Epigen, Betacellulin (BTC), neuregulin-1 (NRG1), neuregulin-2 (NRG2), neuregulin-3 (NRG3), neuregulin-4 (NRG4). In an embodiment, the immature HIOs are cultured in a culture medium that is free or substantially free of exogenous NRG1. In a further embodiment, the immature HIOs are cultured in a culture medium that is free of exogenous NRG1. In an embodiment, the immature HIOs are cultured in a culture medium that is free or substantially free of any exogenous growth factor of the EGF family. In an embodiment, the immature HIOs are cultured in a culture medium that is free of any exogenous growth factor of the EGF family.

The method described herein is advantageously entirely in vitro, i.e., it does not comprise an in vivo step, notably the engraftment of the immature HGIOs (e.g., HIOs) into an animal, as described in Workman et al.⁸.

In an embodiment, the immature HGIOs (e.g., HIOs) are cultured for a period of at least 10 days, at least 14 days (2 weeks), at least 21 days (3 weeks), or at least 28 days (4 weeks). In a further embodiment, the immature HGIOs (e.g., HIOs) are cultured for a period of about 3 weeks to about 6 weeks, or about 4 weeks to about 5 weeks. In an embodiment, the method comprises detaching the organoids from each other during the culture. The detachment may be performed, e.g., every 24 to 72 hours, preferably every 48 hours.

The immature HGIOs (e.g., HIOs) may be cultured in any suitable culture medium that supports cell growth and differentiation. Examples of culture media include Minimum Essential Media (MEM), Dulbecco's Modified Eagle Medium (DMEM), DMEM: Nutrient Mixture F12 (DMEM F12), Roswell Park Memorial Institute (RPMI), Iscove's Modified Dulbecco's Medium (IMDM), and Media 199 (M199 media). The culture medium may be supplemented with any suitable buffers, nutrients, minerals, amino acids, growth factors, preservatives, antioxidants, vitamins, etc. In an embodiment, the culture medium comprises DMEM F12. In an embodiment, the culture medium comprises a B-27™ supplement (Thermo Fisher Scientific, Catalog number: 17504044). In an embodiment, the culture medium comprises at least one amino acid, for example L-glutamine. In an embodiment, the culture medium comprises a physiologically acceptable buffer, for example 4-(2-hydroxyethyl)-1-piperazineethanesulfonic acid (HEPES). In an embodiment, the culture medium comprises one or more antibiotics, such as penicillin, streptomycin or a mixture thereof.

In an embodiment, the conditions suitable for HGIO (e.g., HIO) growth comprise incubation at a temperature of about 36 °C to 38 °C, preferably about 37 °C.

During the culture, the culture medium may be replaced by or supplemented with fresh medium one or more times. In an embodiment, the culture medium is replaced with fresh medium at least every 48 to 96 hours, for example at least every 72 hours or at least every 48 hours.

The term “non-adherent environment” or “non-adherent conditions” as used herein refers to an environment that prevents the adherence of the cells to a solid support such as a culture flask. This may be achieved by various means including:
(1) encapsulation of the HGIO in a low to non-adhesive hydrogel, for example encapsulation in alginate;
(2) culturing of the HGIO in a low to non-adhesive culture surface. This may be achieved by using a surface that is resistant to cellular attachment or modified to reduce cellular attachment. For example, glass, Corning® Costar® Ultra-Low Attachment, or coating the culture surface with 2-hydroxyethyl methacrylate;
(3) Keeping the HGIO in suspension by mechanical movement, for example, by using a bioreactor that creates constant flow of the media containing the HGIO; and/or
(4) Keeping the HGIO in suspension by magnetic force, for example, by coating or incorporating magnetic substances into the HGIO and creating a magnetic field around it to keep it in suspension.

In an embodiment, the immature HGIOs (e.g., HIOs) are cultured in suspension, i.e., in the absence of a matrix that allows cell attachment, such as a biocompatible gel (e.g., Matrigel™ or the like). The immature HGIOs (e.g., HIOs) may be cultured in the absence of any matrix/gel, or in the presence of a matrix/gel that does not allow cell attachment, such as alginate (so that the immature HGIOs (e.g., HIOs) remain in suspension). The immature HGIOs (e.g., HIOs) are cultured in a culture system (e.g., dish, plate or flask) that minimizes or prevents adherence/attachment of the cells to the cultureware surface. The surface of these systems is typically coated with materials that minimize cell attachment, protein absorption, and cellular activation. Examples of culture systems include ultra low attachment (ULA) culture flasks, plates or dishes that are commercially available from various providers (e.g., Thermo Scientific's Nunclon Sphera 3D culture system, Corning's Ultra-Low Attachment Surface, Ultra-Low Adherent Plates for Suspension Culture from STEMCELL Technologies, etc.).

In an embodiment, the immature HGIO (e.g., HIO) has a diameter that is 1 mm or less, preferably a diameter that is 0.9 mm or less, 0.8 mm or less, or 0.7 mm or less.

Immature HGIOs (e.g., HIOs) may be obtained by methods well known in the art as described for example in Workman, M. J. et al.⁸ and McCracken, K. W. et al.⁹ In an embodiment, the HGIOs (e.g., HIOs) are derived from human embryonic or pluripotent stem cells, such as human induced pluripotent stem cells (hiPSC).

The term “pluripotent stem cells (PSCs)” as used herein has its plain and ordinary meaning as understood in light of the specification and encompasses any cells that can differentiate into nearly all cell types of the body, i.e., cells derived from any of the three germ layers (germinal epithelium), including endoderm (interior stomach lining, gastrointestinal tract, the lungs), mesoderm (muscle, bone, blood, urogenital), and ectoderm (epidermal tissues and nervous system). PSCs can be the descendants of inner cell mass cells of the preimplantation blastocyst or obtained through induction of a non-pluripotent cell, such as an adult somatic cell, by forcing the expression of certain genes.

The term “induced pluripotent stem cells (iPSCs),” also commonly abbreviated as iPS cells, as used herein has its plain and ordinary meaning as understood in light of the specification and refers to a type of pluripotent stem cells artificially derived from a normally non-pluripotent cell, such as an adult somatic cell, by inducing a “forced” expression of certain genes. hiPSC refers to human iPSCs. In some methods known in the art, iPSCs may be derived by transfection of certain stem cell-associated genes into non-pluripotent cells, such as adult fibroblasts. Transfection may be achieved through viral transduction using viruses such as retroviruses or lentiviruses. Transfected genes may include the master transcriptional regulators Oct-3/4 (POU5F1) and Sox2, although other genes may enhance the efficiency of induction. After 3-4 weeks, small numbers of transfected cells begin to become morphologically and biochemically similar to pluripotent stem cells, and are typically isolated through morphological selection, doubling time, or through a reporter gene and antibiotic selection. As used herein, iPSCs include first generation iPSCs, second generation iPSCs in mice, and human induced pluripotent stem cells. In some methods, a retroviral system is used to transform human fibroblasts into pluripotent stem cells using four pivotal genes: Oct3/4, Sox2, Klf4, and c-Myc. In other methods, a lentiviral system is used to transform somatic cells with OCT4, SOX2, NANOG, and LIN28. Genes whose expression are induced in iPSCs include but are not limited to Oct-3/4 (POU5F1); certain members of the Sox gene family (e.g., SoxI, Sox2, Sox3, and Sox15); certain members of the Klf family (e.g., Klfl, Klf2, Klf4, and Klf5), certain members of the Myc family (e.g., C-myc, L-myc, and N-myc), Nanog, LIN28, Tert, Fbx15, ERas, ECAT15-1, ECAT15-2, Tcl1, [symbol] -Catenin, ECAT1, Esg1, Dnmt3L, ECAT8, Gdf3, Fth117, Sal14, Rex1, UTF1, Stella, Stat3, Grb2, Prdm14, Nr5a1, Nr5a2, or E-cadherin, or any combination thereof.

In an embodiment, the method for obtaining immature HGIO (e.g., HIO) comprises one or more of the following steps:
(i) culturing pluripotent stem cells in an endoderm differentiation medium supplemented with Activin A until obtention of confluent monolayer of cells;
(ii) culturing the confluent monolayer of cells in a mid-hindgut differentiation medium supplemented with a Fibroblast growth factor (FGF) (e.g., FGF4) and a Wnt pathway activator until obtention of free-floating spheroids;
(iii) mixing the spheroids with a first biocompatible gel supplemented with epidermal growth factor (EGF)
(iv) culturing the gel-spheroid mixture in an intestinal basal medium supplemented with epidermal growth factor (EGF) until obtention of primary intestinal organoids;
(v) mixing the primary intestinal organoids and enteric neural cell precursors with a second biocompatible gel; and
(vi) culturing the mixture of (v) in an intestinal basal medium until obtention of the immature HGIO (e.g., HIO).

In an embodiment, the Wnt pathway activator comprises a Wnt protein, e.g., WntI, Wnt2, Wnt2b, Wnt3, Wnt3a, Wnt4, Wnt5a, Wnt5b, Wnt6, Wnt7a, Wnt7b, Wnt8a, Wnt8b, Wnt9a, Wnt9b, WntlOa, Wntl Ob, Wntl 1, Wntl6, BML 284, IQ-1, WAY 262611, or any combination thereof. In an embodiment, the Wnt pathway activator is an inhibitor of glycogen synthase kinase (GSK) 3 (GSK3β and/or GSK3α). GSK3 is a serine/threonine kinase that is an inhibitor of the WNT pathway; therefore inhibitors of GSK3 functions as a WNT pathway activators. In an embodiment, the Wnt pathway activator comprises CHIR99021, CHIR98014, AZD2858, BIO, AR-A014418, SB 216763, SB 415286, aloisine, indirubin, alsterpaullone, kenpaullone, lithium chloride, TDZD 8, or TWS119, or any combination thereof. In a further embodiment, the Wnt pathway activator is CHIR99021.

In an embodiment, the method for obtaining immature HGIO (e.g., HIO) comprises one or more of the following steps:
passaging confluent hiPSCs in a plate with EDTA and seeding of the cell suspension in plate;
feeding the cell with mTeSR1 daily for two days or until the confluency reached ~80%;
culturing the cell for one day in an endoderm differentiation media base (EDM-base): RPMI1640, pen-strep, nonessential amino acid with Activin A;
culturing the cell for two days in EDM-base supplemented with Activin A and FBS;
culturing the cell for four days in mid-hindgut differentiation media (MHDM): EDM-base, FBS, FGF4, CHIR99021;
collecting the free-floating spheroids and suspending them in Matrigel™ supplemented with B27™ supplement and EGF;
plating the Matrigel™ suspension as a droplet with 15-20 spheroids per droplet on a plate and polymerizing the Matrigel™ at 37 °C;
culturing the spheroids in intestinal basal media (IBMbase): Advanced DMEM-F12, B27, Glutamax™, pen-strep, HEPES supplemented with EGF, Noggin, and Rspondin for six days changing the medium every 48 hours;
culturing the spheroids in IBM supplemented with EGF (IBMe) for two weeks changing the medium every 48 hours;
separating the spheroids from each other and re-embedding them in Matrigel droplets;
mixing the Matrigel™ droplets with vagal neural crest cells (VNCC) in B27 and EGF and polymerizing the Matrigel™ at 37 °C; and
culturing the mixture for 1 week in IBMe.

The VNCC may be derived from hiPSC using methods known in the art such as the methods described in Workman, M. J. et al.⁸ and Bajpai, R. et al.¹⁰. In an embodiment, the method for obtaining VNCC comprises one or more of the following steps:
seeding small colonies of hiPSC at low density (e.g., 1/80 of a confluent well of 6 well plate) and culturing for about 5-6 days;
on the first day of culture, detaching the colonies from the plate (e.g., using collagenase IV);
suspending the colonies in neural induction media (NIM): 1:1 ratio of DMEM-F12 and Neurobasal media, B27, N2, insulin, basic-FGF, EGF, and pen-strep with Rock inhibitor Y27632;
culturing the colonies for about 5-6 days until the neurospheres have clear round borders;
culturing the neurospheres in NIM with all-trans-retinoic acid for about 48 hours;
culturing the neurospheres in NIM in a fibronectin-coated plate for about 6 to 10 days; and
collecting the neurospheres comprising VNCC.

In another aspect, the present disclosure provides a human gastrointestinal organoid (HGIO), such as a human intestinal organoid (HIO), obtained or prepared by the method described herein.

As shown in the examples below, organoids prepared entirely in vitro according to the method described herein mimic the structural/anatomical features of human intestines, including the presence of epithelial villi and crypt, smooth muscle layers, organized interstitial cells of cajal and enteric ganglia, and exhibit peristaltic movement and contractile activity undergo spontaneous contractile activity.

Accordingly, in another aspect, the present disclosure provides a human gastrointestinal organoid (HGIO), such as a human intestinal organoid (HIO), prepared according to the method/process described herein.

The present disclosure also provides a human gastrointestinal organoid (HGIO), such as a human intestinal organoid (HIO), having contractile activity and comprising:
smooth muscle cells,
interstitial cells of cajal,
epithelial cells (e.g., enterocytes, Goblet cells, etc.),
crypt-villi structures, and optionally
enteric neuron cells.

In an embodiment, the HGIO (e.g., HIO) comprises enteric neuron cells.

In an embodiment, the HGIO (e.g., HIO) further comprises connective tissue cells, such as fibroblasts and/or myofibroblasts. In another embodiment, the HGIO (e.g., HIO) further comprises intestinal stem cells.

In an embodiment, the HGIO (e.g., HIO) comprises an inner layer, a mid-layer (or intermediate layer) and an outer layer.

In an embodiment, the inner layer comprises the crypt-villi structures. In an embodiment, the inner layer comprises epithelial cells and intestinal stem cells. In an embodiment, the crypts of the crypt-villi structures (located proximal to the mid-layer) comprise intestinal stem cells. In another embodiment, the villi of the crypt-villi structures comprise epithelial cells (e.g., enterocytes, Goblet cells, etc.).

In an embodiment, the mid-layer comprises connective tissue cells, such as fibroblasts and/or myofibroblasts. In another embodiment, the mid-layer comprises enteric neuron cells.

In an embodiment, the outer layer comprises smooth muscle cells. In another embodiment, the outer layer comprises interstitial cells of cajal. In an embodiment, the outer layer comprises a first (e.g., inner) sublayer adjacent to the mid-layer and a second (e.g., outer) sublayer. In an embodiment, the first (e.g., inner) sublayer comprises smooth muscle cells. In an embodiment, the second (e.g., outer) sublayer comprises interstitial cells of cajal. In another embodiment, the outer layer comprises enteric neuron cells.

In an embodiment, the HGIO (e.g., HIO) comprises:
an inner layer comprising crypt-villi structures comprising epithelial cells and intestinal stem cells and, optionally, intestinal stem cells;
a mid-layer comprises connective tissue cells, such as fibroblasts and/or myofibroblasts and, optionally, enteric neuron cells;
an outer layer comprising (i) a first (e.g., inner) comprising smooth muscle cells and (ii) a second (e.g., outer) sublayer comprising interstitial cells of cajal and, optionally (iii) enteric neuron cells.

In an embodiment, the smooth muscle cells express the marker(s) TAGLN and/or SMTN. In an embodiment, the smooth muscle cells express both TAGLN and SMTN. In an embodiment, the smooth muscle cells are organized in one or more layers. In an embodiment, the thickness of the smooth muscle layer(s) is at least 30 μm. In further embodiments, the thickness of the smooth muscle layer(s) is at least 40 μm, 50 μm or 60 μm. In embodiments, the thickness of the smooth muscle layer(s) is less than 200 μm, less than 175 μm, or less than 150 μm.

In an embodiment, the interstitial cells of cajal (ICC) express the marker KIT (cKIT or CD117).

In an embodiment, the epithelial cells express the marker CDX2. In an embodiment, the epithelial cells face the inside of the HGIO.

In an embodiment, the crypt-villi structures comprise proliferating crypt and non-proliferating villi. In an embodiment, the crypt structures express the marker MKI67.

In an embodiment, the enteric neuron cells express ELAVL4 and/or TUBB3. In a further embodiment, the enteric neuron cells express ELAVL4 and TUBB3. In another embodiment, the enteric neuron cells form ganglia in the HGIO. In an embodiment, the neuronal ganglia have a size of at least 10, 15 or 20 μm. In embodiments, the neuronal ganglia have a size that is less than 60 μm or 50 μm.

In an embodiment, the HGIO have a diameter of about 0.5 mm to about 10 mm. In an embodiment, the HIO have length of about 0.5 mm to about 150 mm.

In an embodiment, the HGIO are obtained by the method defined herein.

In an embodiment, the HGIO are xeno-free, i.e., free of molecules or macromolecules of animal (e.g., mouse) origin, e.g., mouse proteins, nucleic acids, lipids, extracellular matrix, cells, etc.

The HGIO according to the present disclosure may be used for various applications, for example for studying and assessing human intestinal development, homeostasis, disease, response to drugs, and regeneration.

In another aspect, the present disclosure provides a method or screening assay for determining whether a test agent modulates an activity and/or function of intestines comprising: contacting the HGIO defined herein with the test agent; and determining the activity and/or function of the HGIO in the presence and absence of the test agent, wherein a difference in the activity and/or function of the HGIO in the presence vs. the absence of the test agent is indicative that the test agent modulates the activity and/or function of intestines.

As used herein, the term “test agent” may be any type of agents/molecules including small compounds, antibodies, nucleic acids, peptides, proteins, cells, and extracts or natural products. The above-noted screening assay may be applied to a single test agent or to a plurality or “library” of such agents (e.g., a combinatorial library).

Screening assay systems may comprise a variety of means to enable and optimize useful assay conditions. Such means may include but are not limited to: suitable buffer solutions, for example, for the control of pH and ionic strength and to provide any necessary components for optimal activity and stability (e.g., protease inhibitors), temperature control means for optimal activity and/or stability of the components (HGIO, test agents), and detection means to enable the detection of the activity and/or function of the HGIO.

In an embodiment, the activity and/or function is contractile or motile activity. In an embodiment, the method comprises measuring electromagnetic radiation coming from the direction of the organoid, such as visible light, and assessing the changes in the array of data acquired (using camera to record contraction). In another embodiment, the method comprises measuring the electric field generated by the organoid, and assessing the changes in the array of data acquired (using microelectrode array to measure electric field).

The screening method of the present disclosure may be usefully applied to searching and developing prophylactic or therapeutic agents for gastrointestinal-related diseases.

### EXAMPLES

The present disclosure is illustrated in further details by the following non-limiting examples.

### Example 1: Materials and Methods

### Pluripotent Stem Cell Culture

Human induced pluripotent stem cells (hiPSC) were cultured with a feeder-free maintenance medium for human ES and iPS cells (mTeSR™1, StemCell Technologies, Cat. No. 85850) with 1× penicillin-streptomycin and hESC-qualified Matrigel™ (354277, Corning). Matrigel™ was coated onto Nunc Delta surface plates (14-832-11, Thermo Scientific) as per manufacturer recommendation. The cells were passaged as small clusters using 0.5 mM ethylenediaminetetraacetic acid (EDTA) in phosphate buffered saline (PBS). The cells were cryopreserved with NutriFreez™D10 (05-713-1E, Biological Industries) as per manufacturer recommendation.

### Human Intestinal Organoid Derivation

Human intestinal organoids (HIO) can be derived with the hiPSC or hPSC as previously described with minor modifications^{8,9}. Briefly, 85% confluent hiPSCs in a 6-well plate were passaged with EDTA and seeded 1/14- 1/16 of a cell suspension per single well of 24-well plate. The cells were fed with mTeSR 1 daily for two days or until the confluency reached 80%. On the first day, the media was changed with endoderm differentiation media base (EDM-base): RPMI1640 (11875-093, Gibco), 1× pen-strep (15015067, Wisent), 1× nonessential amino acid (11140050, Gibco) with 100 ng/ml Activin A (338AC010, R&D systems). On the second day, the cells were fed EDM-base supplemented with 100 ng/ml Activin A and 0.2% FBS (HyClone™, Fisher Scientific). On the third day, the cells were fed the same as the second but with 2% FBS. The fourth day, the confluent monolayer of cells were fed with mid-hindgut differentiation media (MHDM): EDM-base, 2% FBS, 500 ng/ml FGF4(235F4025 CF, R&D systems), 3 μM CHIR99021 (S1263, Selleckchem). The cells were fed MHDM daily for a total of 4 days. At the end of mid-hindgut differentiation, the free-floating spheroids were collected and suspended in Matrigel™ (354234, Corning) supplemented with 1× B27™ supplement (17504044, Gibco) and 100 ng/ml EGF (236EG200, R&D systems; AF-100-15, PeproTech). The Matrigel™ suspension were plated as a droplet with 15-20 spheroids per droplet on a plate (14-832-11 or 130184, Thermo Scientific) and polymerized at 37 °C for 10 min. The spheroids were fed intestinal basal media (IBMbase): Advanced DMEM-F12 (12634-010, Gibco), 1× B27 (17504044, Gibco), 1× Glutamax™ (35050061, Gibco), 1× pen-strep, 15 mM HEPES (15630080, Gibco) supplemented with 100 ng/ml EGF, 100 ng/ml Noggin, and 100 ng/ml R-spondin (IBMrne) for six days changing the medium every 48 hours. They were then fed IBM supplemented with 100 ng/ml EGF (IBMe) every 48 hours. Two weeks after the spheroid collection, the organoids were passaged by manually separating them from each other with sterile syringe needle and re-embedding in the Matrigel™ droplet as before. At this point, to include enteric neurons, vagal neural crest cells (VNCC) were combined as follows.

### Vagal Neural Crest Combination

Matrigel™ was supplemented to 1× B27™ and 100 ng/ml EGF. In a 5 ml tube, 50,000 VNCC were added to maximum x15 HIOs. Optimal number of VNCC may vary depending on the cell line. The mix was briefly triturated using a 1000 μl pipette-tip, kept at −20 °C, where 1 mm of the end of the tip was cut. The mix was then centrifuged at 300 g for 3 min. The HIOs and the cells were then gently resuspended and centrifuged again. Supernatant was removed as much as possible and 50 μl of cold Matrigel™ was added. The tube was kept on a cold tube from this point on. Using a cut-pipette tip that is cold, HIO and cells were gently resuspended, and the total volume was gently pipetted as a droplet on a pre-warmed 6-well plate. Up to four droplets were plated per well. The plate was gently transferred to the incubator and allowed to polymerize for 10 min. The well was then filled with 2 ml of IBMe. The medium was refreshed every 48 hr. One week after the co-culture, the organoids were removed from the Matrigel™ with gentle trituration and dissection with sterile syringe needles then proceeded to organoid maturation procedure.

### Vagal Neural Crest Derivation

Vagal neural crest cells (VNCC) were derived from the hiPSC as previously described with minor modifications^{8,10}. Briefly, in a 6-well plate, small colonies of hiPSC were seeded at low density ( 1/80 of a confluent well of 6-well plate) and grown for 5 days. On the first day of differentiation, the colonies were lifted with 500 U/ml collagenase IV (17104019, Gibco) in mTeSR1 for up to 1 hr in the incubator or until the colonies detached completely with gentle taps to the plate. The colonies were washed with 2 ml of DMEM/F12 (319-075-CL, Wisent) three times. They were then suspended in neural induction media (NIM): 1:1 ratio of DMEM-F12 and Neurobasal media (21103049, Gibco), 0.5× B27, 0.5× N2, 5 μg/ml insulin (12643, Sigma), 20 ng/ml basic-FGF (100-18B, PeproTech), 20 ng/ml EGF, and 1× pen-strep with 5 μM of Rock inhibitor Y27632 (S1049, Selleckchem) and transferred to non-tissue culture treated plate (08-772-51, Fisher scientific). The NIM was changed daily with decreasing Y27632 (Y27) concentration (Day 2: 2.5 μM Y27, Day 3 and beyond: no Y27), for additional 5 to 6 days until the neurospheres had clear round borders. The neurospheres were then fed NIM with 2 μM all-trans-retinoic acid (R2625, Sigma Aldrich) daily for two days. The neurospheres were transferred on to the fibronectin (PHE0023, Gibco) coated plate in NIM (w/o retinoic acid) and left undisturbed for 48 hours. Fibronectin coated plates were prepared by incubating plastic tissue culture plates with 15 μg/ml fibronectin in PBS without calcium or magnesium at 37 °C overnight. Afterwards, the NIM was changed daily until neural crest cells migrated and spread out onto the plate (6-10days). Neurospheres were mechanically removed and the migrated neural crest cells were lifted as single cells with a 5-minute incubation at 37 °C with 1× TrypLE (A1217701, Gibco). Cells were washed by diluting with 9 ml of room temperature DMEM/F12 and centrifuging at 300 g for 4 min. The supernatant was removed and cells were resuspended in DMEM/F12 for co-culture with HIO or resuspended in NIM and plated back onto fibronectin coated plate and maintained until the experiment. The cells were positive for known vagal fate neural crest cell markers and gene expression.

### Organoid In Vitro Maturation

The starting material was two-to-four-week HIO or HIO/ENS that were no larger than 0.7 mm in diameter. 2 ml of prewarmed IBMe was added to each well of ULA 6-well plate. Organoids were separated from the Matrigel™ and transferred into a single well of ULA 6-well plate. After 48 hours, the organoids were detached from each other if they had stuck together. The media was completely replaced with 2 ml of prewarmed half/half mix of IBMbase and IBMe. After 48 hours, the organoids were detached from each other if they had stuck together and the media was completely replaced with 2 ml of prewarmed IBMbase. Organoids were continuously detached from each other and fresh IBMbase was added for 4-5 weeks. The organoids were starting to show spontaneous contractile activity in two weeks.

### Measurement of Contractile Activity

Time-lapse images of HIOs were taken in temperature and humidity-controlled environment. The video file was then loaded and analyzed with ImageJ/Fiji using Muscle motion package.

### Differentiation of Mesenchymal Cells of Human Intestinal Organoid Into Smooth Muscle Cells

Mesenchymal outgrowth from HIO was dissected from the Matrigel™ droplet and dissociated in TrypLE1× for 10 min. The resulting cell suspension was passed through a 70 μM filter and diluted ten-fold in PBS and centrifuged at 300 g for 4 min. The resulting pellet was resuspended in experimental media conditions and plated on a 24 well plate with Nunc delta coated plastic coverslips. Standard immunocytochemistry was performed.

### Example 2: Results

One of the primary challenges of prolonged in vitro culture of pluripotent stem cell derived tissue, or organoid, with the intent to further mature it further down the developmental timeline, comes with its increase in size. Nutrient/gas exchange diminishes towards the center of the organoid as it grows, and vascularization is required to overcome this limitation. Many efforts are being made to engineer effective means of in vitro vascularization¹⁻³; however, the present inventors took an alternate approach where they kept the human intestinal organoid (HIO) intentionally small and simultaneously facilitated its maturation or differentiation.

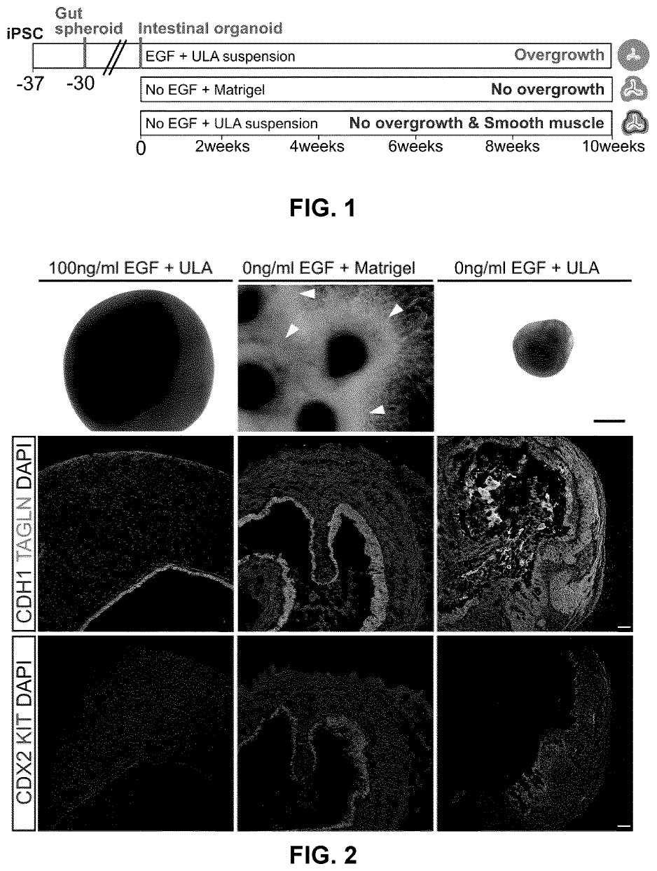

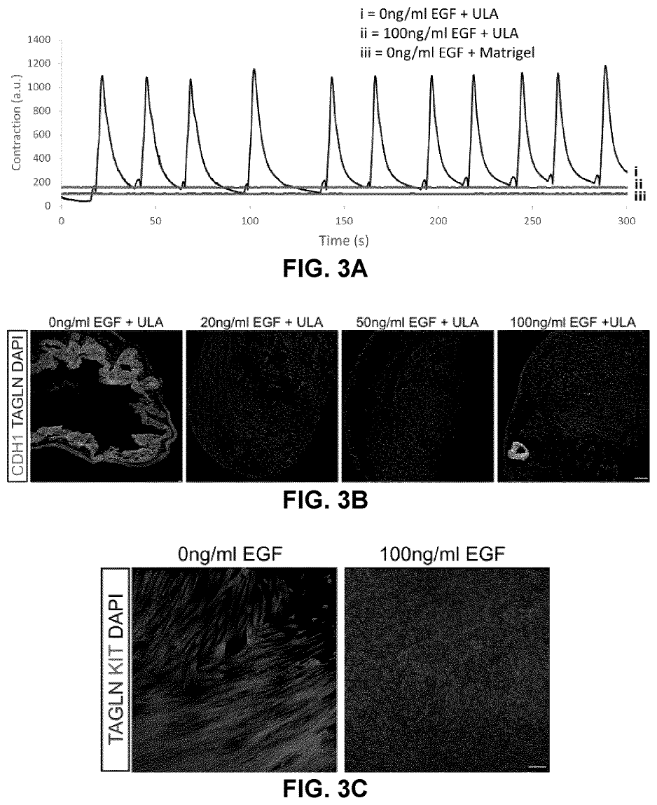

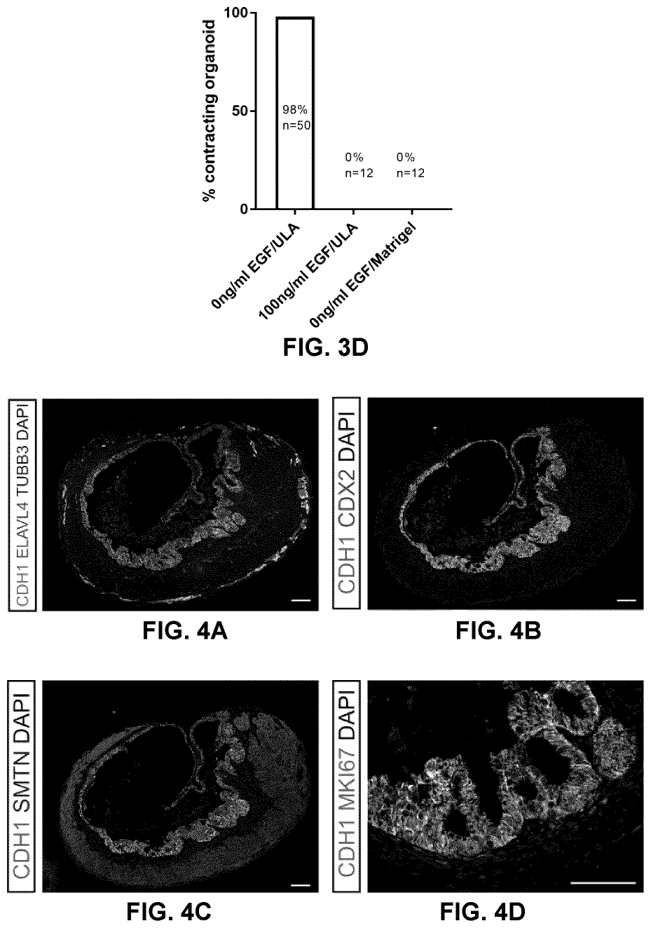

One of the most noticeable biochemical differences between the in vitro culture condition and grafted state is “high” growth factor concentrations. EGF is a potent cellular trophic factor and is commonly included in epithelial organoid cultures at high concentration (e.g., 100 ng/ml) to promote cell proliferation^{4,5}. However, previous reports also indicate that EGF inhibits the differentiation of mesodermal progenitors into smooth muscle cells^{6,7}. It was thus hypothesized that high EGF level in the HIO medium was responsible for its uninhibited “overgrowth” and was simultaneously inhibiting the progression of mesodermal mesenchymal cells from differentiating into smooth muscle cells. Week 4 HIOs were cultured in medium without added/exogenous EGF in Matrigel™ for up to 70 days (FIGS. 1, 2). HIO grown without added/exogenous EGF showed only minor growth, though mesenchymal cells continued to expand within the Matrigel™, retained CDX2 expression in the epithelium, an intestine specific transcription factor. However, it did not show smooth muscle development (TAGLN-positive) or contractile activity (FIGS. 2, 3A, D).

Many previous organoid cultures, including HIO cultures, use extracellular matrix such as Matrigel™ to maintain organoid in a 3-dimensional environment. However, neither the developing intestine nor the grafted HIO, which do mature further, require to be enveloped in soft protein matrix in order to mature. Developing intestine is enclosed by mesodermal derived serosal cells which acts as the organ's boundary, thus, in essence, the intestine is suspended in the abdominal cavity with only itself to physically interact with. It was assessed whether such biophysical environment can be mimicked by suspending the HIO in ultra-low attachment (ULA) plate at week 4 after the mesenchymal cells has established around the epithelial cells. The HIOs were cultured in medium containing either 100 ng/ml EGF or 0 ng/ml added/exogenous EGF in ULA plate. Interestingly, HIO in 0 ng/ml added/exogenous EGF medium in ULA started to undergo spontaneous contractile activity as early as two weeks into the new condition and almost all HIOs underwent peristalsis-like activity by fourth week and the peristalsis-like contractile activity continue up to our longest culture period of 70 days (FIG. 3A, D). On the other hand, HIO in 100 ng/ml EGF level in ULA did not show any contractile activity and developed thick layer of mesenchymal layer. Most HIOs cultured in 100 ng/ml conditions no longer possessed epithelial cells (FIGS. 2, 3A, D). Further histological analyses revealed that HIO in 0 ng/ml EGF/ULA condition developed TAGLN-positive smooth muscle structures and KIT-positive interstitial cells of cajal (ICC), known to regulate intestinal contraction (FIG. 2). The epithelial cells were positive for CDX2, and HIOs developed crypt-villi structures. Neither the HIOs in 100 ng/ml EGF/ULA or 0 ng/ml EGF/Matrigel™ conditions developed TAGLN-positive smooth muscle structures, and KIT-positive cells were rarely detected (FIG. 2).

In order to further define the effect of EGF in maturation, HIOs were cultured in varying EGF concentrations suspended in ULA. Overgrowth of mesenchymal cells were observed in 20, 50, and 100 ng/ml concentrations, but not in 0 ng/ml EGF condition. No sign of further maturation, such as smooth muscle or spontaneous contractions, were observed in in 20, 50, and 100 ng/ml, in contrast to the 0 ng/ml EGF condition (FIG. 3B).

To test if the smooth muscle differentiation was dependent on the presence of epithelial population, mesenchymal cells were collected from the HIO and cultured on tissue culture treated plates with 0 ng/ml EGF medium or 100 ng/ml EGF added medium for 50 days. Mesenchymal cells in 0 ng/ml EGF differentiated into TAGLN-positive smooth muscle cells, contrary to mesenchymal cells cultured in the presence of 100 ng/ml EGF. No contractile activity or KIT-positive cells, however, were found in either condition (FIG. 3C).

Enteric nervous system (ENS) is important for normal peristaltic activity. Previous study has shown enteric nervous system development when HIO is incorporated with vagal neural crest cells and transplanted in immunodeficient mice. It was tested whether the incorporation of vagal neural crest into HIO would result in enteric nervous system development with the in vitro maturation condition described herein (0 ng/ml EGF in suspension). The combined vagal neural crest cells differentiated into ganglionic neurons and could be observed on day-35 organoids. The organoids again retained intestinal fate (CDX), smooth muscle (SMTN), and crypt-villi structure (MKI67) in this condition (FIG. 4). Thus, it may be concluded that HIO with developed ENS (HIO/ENS) can be produced under the in vitro maturation conditions described herein.

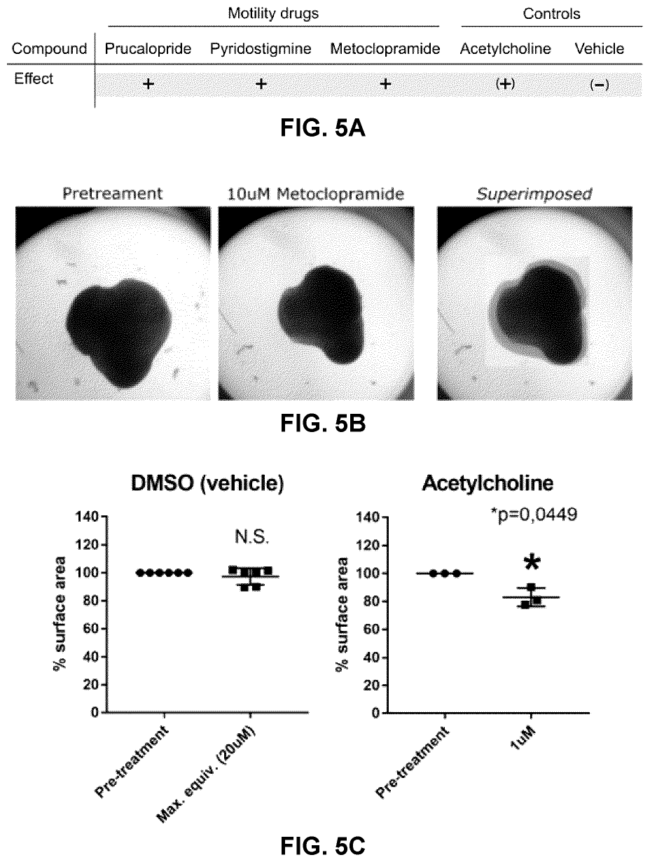

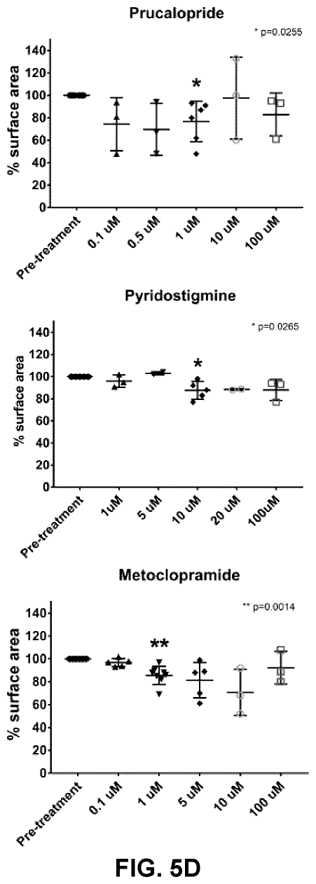

In order to assess the functionality of the smooth muscle, enteric neurons and associated motility observable in the said organoid, three drugs that are used to promote intestinal motility by stimulation of the enteric nervous system (prucalopride, pyridostigmine, and metoclopramide) were used to test if contractility of the organoids can be modified. Within 10 min of treatment with a positive control acetylcholine, which induces muscle contraction, the size of the organoid decreased due to sustained muscle contraction. This decrease in size was absent in organoids treated with vehicle (dimethyl sulfoxide). Treatment with prucalopride, pyridostigmine, and metoclopramide each showed decrease in size in a dose-dependent manner, indicating sustained smooth muscle contraction (FIG. 5).

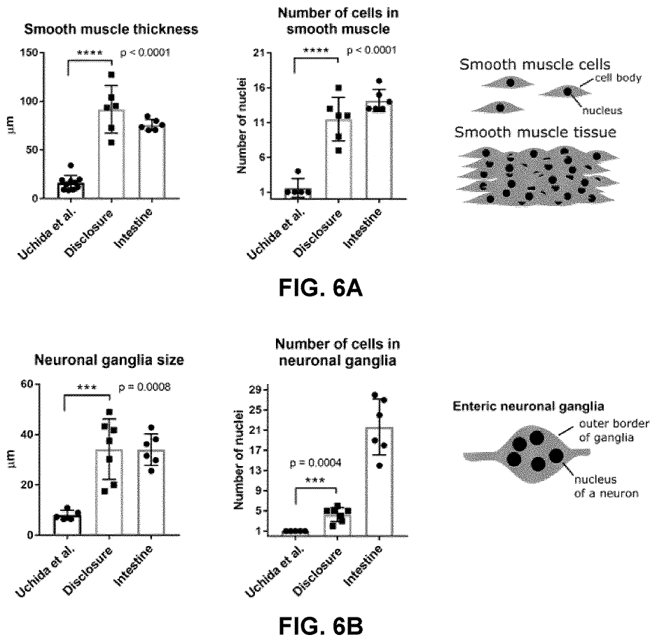

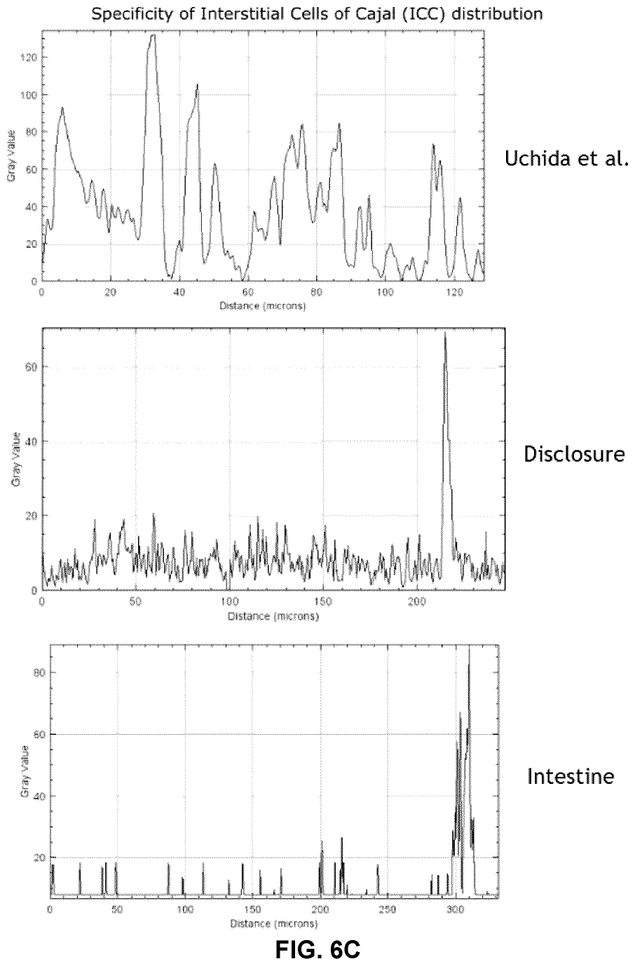

A comparison of specific features of the HIOs obtained according with the process described herein with those of HIOs obtained by the process disclosed in Uchida et al.¹² is presented in FIGS. 6A-C. Relative to HIOs obtained by the process disclosed in Uchida et al.¹² (Uchida et al.), HIOs obtained according with the process described herein (Disclosure) have thicker smooth muscle layers (FIG. 6A, left bar graph) and higher numbers of smooth muscle cells (FIG. 6A, right bar graph), similar to fetal human intestine (Intestine). Furthermore, the neuronal ganglia were significantly bigger (FIG. 6B, left bar graph) and the number of cells in neuronal ganglia was significantly higher in HIOs obtained by the process described herein relative to HIOs obtained by the process disclosed in Uchida et al. Finally, the results depicted in FIG. 6C show that HIOs obtained by the process described herein exhibit a more spatially specific distribution within the tissue, similar to the fetal human intestine.

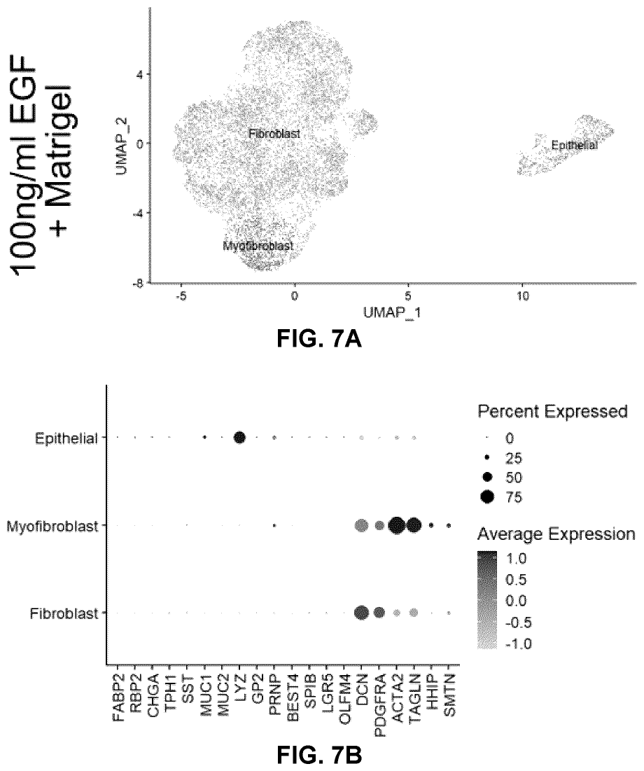

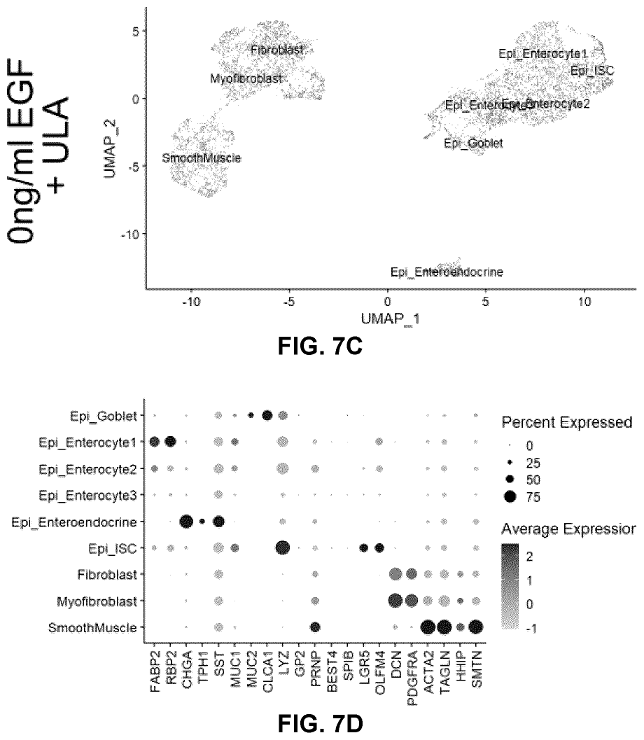

Single-cell RNA sequencing showed that HIO cultured in the absence of EGF (0 ng/ml EGF) in ULA further developed to contain differentiated cell types required for human gut function (FIGS. 7C-D). Specifically, subtypes of intestinal epithelial cells such as goblet (MUC1, MUC2, CLCA1), enterocyte (FABP2, RBP2), enteroendocrine (CHGA, TPH1, SST), and intestinal stem cell niche cells (LYZ, LGR5, OLFM4) were identified function (FIGS. 7C-D). Fibroblast and myofibroblast (DCN, PDGFRA) were also identified function (FIGS. 7C-D). Finally, mature non-vascular smooth muscle cells of muscularis layer (ACTA2, TAGLN, HHIP, SMTN) were present function (FIGS. 7C-D). In comparison, HIO in the condition 100 ng/ml EGF in Matrigel™, did not show any signs of further differentiated cell type function (FIGS. 7A-B). Specifically, there were no noticeable gene expression indicating the presence of epithelial subtypes (FIGS. 7A-B). Gene expression indicating the presence of non-vascular smooth muscle was not observed (FIGS. 7A-B). Thus, the transcriptomic analysis also indicates that the HIO in 0 ng/ml EGF in ULA has matured and differentiated significantly over the 100 ng/ml in Matrigel™.

Although the present invention has been described hereinabove by way of specific embodiments thereof, it can be modified, without departing from the spirit and nature of the subject invention as defined in the appended claims. In the claims, the word “comprising” is used as an open-ended term, substantially equivalent to the phrase “including, but not limited to”. The singular forms “a”, “an” and “the” include corresponding plural references unless the context clearly dictates otherwise.

### REFERENCES

1. Kress, S. et al. Evaluation of a Miniaturized Biologically Vascularized Scaffold in vitro and in vivo. Sci. Rep. 8, (2018).
2. Grigoryan, B. et al. Multivascular networks and functional intravascular topologies within biocompatible hydrogels. Science, Vol. 364, No. 6439, pp. 458-464, (2019).
3. Wimmer, R. A., Leopoldi, A., Aichinger, M., Kerjaschki, D. & Penninger, J. M. Generation of blood vessel organoids from human pluripotent stem cells. Nat. Protoc. Vol. 14, No. 11, doi:10.1038/s41596-019-0213-z. (2019).
4. Sato, T. et al. Single Lgr5 stem cells build crypt-villus structures in vitro without a mesenchymal niche. Nature 459(7244):262-5 doi:10.1038/nature07935. (2009).
5. Sato, T. et al. Long-term expansion of epithelial organoids from human colon, adenoma, adenocarcinoma, and Barrett's epithelium. Gastroenterology 141, 1762-1772 (2011).
6. Kimura, H. et al. EGF positively regulates the proliferation and migration, and negatively regulates the myofibroblast differentiation of periodontal ligament-derived endothelial progenitor cells through MEK/ERK-and JNK-dependent signals. Cell. Physiol. Biochem. 32, 899-914 (2013).
7. Leroy, M. C., Perroud, J., Darbellay, B., Bernheim, L. & Konig, S. Epidermal Growth Factor Receptor Down-Regulation Triggers Human Myoblast Differentiation. PLoS ONE 8, (2013).
8. Workman, M. J. et al. Engineered human pluripotent-stem-cell-derived intestinal tissues with a functional enteric nervous system. Nat. Med. 23, pages 49-59, doi:10.1038/nm.4233. (2017)
9. McCracken, K. W., Howell, J. C., Wells, J. M. & Spence, J. R. Generating human intestinal tissue from pluripotent stem cells in vitro. Nat. Protoc. 6, 1920-1928 (2011).
10. Bajpai, R. et al. CHD7 cooperates with PBAF to control multipotent neural crest formation. Nature 463, 958-962 (2010).
11. Carey L Watson, Maxime M Mahe, Jorge Munera, Jonathan C Howell, Nambirajan Sundaram, Holly M Poling, Jamie I Schweitzer, Jefferson E Vallance, Christopher N Mayhew, Ying Sun, Gregory Grabowski, Stacy R Finkbeiner, Jason R Spence, Noah F Shroyer, James M Wells, Michael A Helmrath. An in vivo model of human small intestine using pluripotent stem cells. Nat Med. 2014 Nov;20(11):1310-4. doi: 10.1038/nm.3737. Epub 2014 October 19.
12. Hajime Uchida, Masakazu Machida, Takumi Miura, Tomoyuki Kawasaki, Takuya Okazaki, Kengo Sasaki, Seisuke Sakamoto, Noriaki Ohuchi, Mureo Kasahara, Akihiro Umezawa, and Hidenori Akutsu. A xenogeneic-free system generating functional human gut organoids from pluripotent stem cells. JCI Insight. 2017; 2(1): e86492

1-42. (canceled)

43. An in vitro method for generating human gastrointestinal organoid (HGIO) having contractile activity comprising:
providing an immature HGIO;
culturing the immature HGIO in a non-adherent environment in a culture medium that is free or substantially free of epidermal growth factor (EGF) under conditions suitable for HGIO growth for a period of at least 1 week, thereby obtaining (HGIO) having contractile activity; and
collecting the HGIO.

44. The method of claim 43, wherein the culture medium that is free or substantially free of any growth factor of the EGF family.

45. The method of claim 43, wherein the culturing is for a period of at least two weeks.

46. The method of claim 43, wherein the culture medium comprises Dulbecco's Modified Eagle Medium/Nutrient Mixture F-12, and optionally a B-27 supplement.

47. The method of claim 46, wherein the culture medium further comprises a source of L-glutamine, 4-(2-hydroxyethyl)-1-piperazineethanesulfonic acid (HEPES), one or more antibiotics, or any combination thereof.

48. The method of claim 43, wherein the conditions suitable for HIO growth comprise incubation at a temperature of about 37 °C.

49. The method of claim 43, wherein the culture medium is replaced with fresh medium at least every 48 to 96 hours.

50. The method of claim 43, wherein the culturing is performed in an ultra low attachment (ULA) culture flask.

51. The method of claim 43, wherein the immature HGIO has a diameter that is 1 mm or less.

52. The method of claim 43, wherein the immature HIO are derived from embryonic stem cells, pluripotent stem cells or induced pluripotent stem cells (iPSC).

53. The method of claim 43, further comprising generating the immature HGIO.

54. The method of claim 53, wherein generating the immature HGIO comprises:
(i) culturing pluripotent stem cells in an endoderm differentiation medium supplemented with Activin A until obtention of confluent monolayer of cells;
(ii) culturing the confluent monolayer of cells in a mid-hindgut differentiation medium supplemented with a Fibroblast growth factor (FGF) and a Wnt pathway activator until obtention of free-floating spheroids;
(iii) mixing the spheroids with a first biocompatible gel supplemented with epidermal growth factor (EGF);
(iv) culturing the gel-spheroid mixture in an intestinal basal medium supplemented with EGF until obtention of primary gastrointestinal organoids;
(v) mixing the primary gastrointestinal organoids and enteric neural cell precursors with a second biocompatible gel; and
(vi) culturing the mixture of (v) in an intestinal basal medium supplemented with EGF until obtention of the immature HGIO.

55. The method of claim 54, wherein (a) the FGF is FGF4, (b) the Wnt pathway activator is CHIR99021, (c) the first and/or second biocompatible gel comprises extracellular matrix, (d) the first and/or second biocompatible gel is a solubilized basement membrane matrix secreted by Engelbreth-Holm-Swarm (EHS) mouse sarcoma cells (Matrigel™), or (e) any combination of (a) to (d).

56. An in vitro human gastrointestinal organoid (HGIO) having contractile peristaltic-like activity and comprising:
smooth muscle cells,
interstitial cells of cajal,
epithelial cells,
crypt-villi structures, and optionally
enteric neuron cells.

57. The HGIO of claim 56, wherein the HGIO comprises:
an inner layer comprising the crypt-villi structures comprising epithelial cells and intestinal stem cells;
a mid-layer comprising connective tissue cells, and, optionally, enteric neuron cells;
an outer layer comprising (i) a first comprising smooth muscle cells and (ii) a second sublayer comprising interstitial cells of cajal and, optionally (iii) enteric neuron cells.

58. The HGIO of claim 56, wherein (a) the smooth muscle cells express TAGLN and/or SMTN, (b) the interstitial cells of cajal express KIT, (c) the epithelial cells express CDX2; (d) the crypt structures express MKI67; (e) the enteric neuron cells express ELAVL4 and/or TUBB3, or (f) any combination of (a) to (e).

59. The HGIO of claim 56, wherein the epithelial cells face the inside of the HGIO.

60. The HGIO of claim 56, wherein the enteric neuron cells form ganglia and/or the smooth muscle cells form one or more layers.

61. The HGIO of claim 56, wherein the HGIO is a human intestinal organoid (HIO).

62. The HGIO of claim 56, wherein the HGIO are obtained according to a method comprising:
providing an immature HGIO;
culturing the immature HGIO in a non-adherent environment in a culture medium that is free or substantially free of epidermal growth factor (EGF) under conditions suitable for HGIO growth for a period of at least 1 week, thereby obtaining (HGIO) having contractile activity; and
collecting the HGIO.
